(19) United States
(12) Patent Application Publication
(10) Pub. No.: US 2014/0214087 A1
(43) Pub. Date: Jul. 31, 2014

(54) JOINT ALIGNER IMPLANT

(71) Applicant: SOLANA SURGICAL, LLC, Memphis, TN (US)

(72) Inventors: Rebecca H. Wahl, Escondido, CA (US); Bruce R. Lawrence, Oceanside, CA (US)

(73) Assignee: SOLANA SURGICAL, LLC, Memphis, TN (US)

(21) Appl. No.: 13/836,979

(22) Filed: Mar. 15, 2013

Related U.S. Application Data
(60) Provisional application No. 61/758,124, filed on Jan. 29, 2013.

(51) Int. Cl. A61B 17/80

(52) U.S. Cl. CPC A61B 17/8061
USPC 606/281; 606/286

(57) ABSTRACT

A joint aligner (12) for use with a flexible first connector (26) for aligning a joint (18) between a first bone (14) and a second bone (16) comprises an aligner body (20) that is adapted to be secured to the first bone (14), the aligner body (20) including a first connector aperture (22) that is adapted to receive the first connector (26) so that the first connector (26) can extend between the first connector aperture (22) and bone connecting material (19) to couple the first bone (14) to at least a portion of the bone connecting material (19). Additionally, the first connector aperture (22) can be positioned along an outer surface (232) of the aligner body (20) and can extend at an aperture angle (250) of between approximately fifteen and twenty-five degrees relative to a first end (228).

### RELATED INVENTION

This application claims priority on U.S. Provisional Application Ser. No. 61/758,124, filed Jan. 29, 2013 and entitled “JOINT ALIGNER PLATE IMPLANT”. As far as permitted, the contents of U.S. Provisional Application Ser. No. 61/758,124 are incorporated herein by reference.

### BACKGROUND

In the human body, joints exist where a first bone contacts or is coupled to a second bone. Additionally, the joints can be held together by certain bone connecting material. Such bone connecting material within and/or adjacent to a joint can include cartilaginous material and ligaments. For example, in the human foot, the plantar plate is a fibrocartilaginous structure found in the joint between the metatarsal and the proximal phalanx, i.e. the metatarsophalangeal (MTP) joint, and in the joints between the distal phalanx and the middle phalanx and between the middle phalanx and the proximal phalanx, i.e. the interphalangeal (IP) joints. The anatomy and composition of the plantar plates is such that the proximal origin is thin, but the distal insertion is stout. At the MTP joint, the plantar plate plays an important role in the foot's weight-bearing function. Unfortunately, trauma, inflammation, instability and/or malalignment (also referred to as misalignment) in the MTP joint, i.e. between the metatarsal and the proximal phalanx, of the toes can lead to discomfort or certain deformities within the toes of the foot, e.g., crossover toe, where the second toe crosses under or over the big toe. Thus, it is desired to maintain proper alignment within the MTP joint to inhibit such discomfort and deformities within the toes of the foot.

### SUMMARY

The present invention is directed toward a joint aligner for use with a flexible first connector for aligning a joint between a first bone and a second bone, the joint including bone connecting material that is connected to at least the second bone. In certain embodiments, the joint aligner comprises an aligner body that is adapted to be secured to the first bone, the aligner body including a first connector aperture that is adapted to receive the first connector so that the first connector can extend between the first connector aperture and the bone connecting material to couple the first bone to at least a portion of the bone connecting material.

In one embodiment, the aligner body includes a first end and an opposed second end, and the first connector aperture extends at an aperture angle of between approximately fifteen and twenty-five degrees relative to the first end. In such embodiment, the first connector aperture is adapted to receive the first connector so that the first connector can extend between the first connector aperture and the bone connecting material to couple the first bone to at least a portion of the bone connecting material.

Additionally, in one embodiment, the aligner body includes a first end and an opposed second end, and the first connector aperture extends substantially perpendicular to the first end. In such embodiment, the first connector aperture is adapted to receive the first connector so that the first connector can extend between the first connector aperture and the bone connecting material to couple the first bone to at least a portion of the bone connecting material.

In some embodiments, the first connector aperture is positioned along an outer surface of the aligner body.

In certain embodiments, the aligner body can further include a second connector aperture that is adapted to receive a flexible second connector so that the second connector can extend between the second connector aperture and the bone connecting material to couple the first bone to at least a portion of the bone connecting material, the second connector aperture being positioned along the outer surface of the aligner body.

Additionally, the aligner body can further include a second connector aperture that is adapted to receive the first connector so that the first connector can extend from the first connector aperture to the bone connecting material and to the second connector aperture to couple the first bone to at least a portion of the bone connecting material.

In one embodiment, the aligner body has a substantially C-shaped cross-section.

In some embodiments, the aligner body further includes a fixation aperture that is adapted for receiving a fixation attacher so that the fixation attacher can fixedly secure the aligner body to the first bone.

Additionally, in certain embodiments, the aligner body is tapered at a taper angle from a first end toward a second end. In some such embodiments, the taper angle is between approximately twelve and twenty-four degrees.

In one embodiment, the first bone is a proximal phalanx, the second bone is a metatarsal, and the joint is a metatarsophalangeal joint between the proximal phalanx and the metatarsal. Additionally, the bone connecting material can be a plantar plate. In such embodiment, the aligner body is secured to the proximal phalanx, the first connector aperture is angled from dorsal distal to plantar proximal so that the first connector can extend between the first connector aperture and the plantar plate to couple the proximal phalanx to the plantar plate.

The present invention is further directed toward a combination including the joint aligner as described above, and a flexible first connector that extends between the first connector aperture and the bone connecting material to couple the first bone to at least a portion of the bone connecting material. In one such embodiment, the aligner body further includes a second connector aperture that is adapted to receive the first connector, the first connector extending from the first connector aperture to the bone connecting material and back to the second connector aperture to couple the first bone to at least a portion of the bone connecting material.

In another application, the present invention is directed toward a method for aligning a joint between a first bone and a second bone, the joint including bone connecting material that is connected to at least the second bone, the method comprising the step of securing an aligner body to the first bone, the aligner body including a first connector aperture that is adapted to receive the first connector so that the first connector can extend between the first connector aperture and the bone connecting material to couple the first bone to at least a portion of the bone connecting material.

Additionally, in still another application, the present invention is directed toward a joint aligner for use with a flexible first connector and a flexible second connector for aligning a joint between a first bone and a second bone, the joint including cartilaginous material and a ligament that are connected to at least the second bone, the joint aligner comprising an aligner body that is adapted to be secured to the first bone, the aligner body including: (i) a first end; (ii) an opposed second end; (iii) a first connector aperture that extends at an aperture angle of between approximately fifteen and twenty-five degrees relative to the first end, the first connector aperture that is adapted to receive the first connector so that the first connector can extend between the first connector aperture and the cartilaginous material to couple the first bone to the cartilaginous material; and (iv) a second connector aperture that extends substantially perpendicular to the first end, the second connector aperture being adapted to receive the second connector so that the second connector can extend between the second connector aperture and the bone connecting material to couple the first bone to at least the ligament.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a top perspective view of a portion of a foot, an embodiment of a joint aligner implant having features of the present invention implanted therein, and a first connector and a second connector that are usable with the joint aligner;

FIG. 1B is a simplified side view of a portion of the foot, and the joint aligner and the first connector of FIG. 1A;

FIG. 1C is a simplified side view of a portion of the foot, and the joint aligner and the second connector of FIG. 1A;

FIG. 2A is a perspective view of another embodiment of the joint aligner implant illustrated in FIG. 1;

FIG. 2B is another perspective view of the joint aligner implant of FIG. 2A;

FIG. 2C is a top view of the joint aligner implant of FIG. 2A;

FIG. 2D is an end view of the joint aligner implant of FIG. 2A;

FIG. 2E is a side view of the joint aligner implant of FIG. 2A;

FIG. 2F is another end view of the joint aligner implant of FIG. 2A; and

FIG. 3 is a perspective view of a portion of the foot and yet another embodiment of a joint aligner implant having features of the present invention implanted therein.

### DESCRIPTION

The present invention is directed to a joint aligner implant (also referred to herein simply as a “joint aligner”) that can be used to maintain proper alignment between adjacent bones and/or within a joint in the human body. For example, various embodiments of the joint aligner can be effectively utilized to maintain proper alignment between the metatarsal and the proximal phalanx within the MTP joint in the toes of the human foot, e.g. within the second, third, fourth or fifth toes of the foot. It should be noted that although the present invention is illustrated and described herein as being used within an MTP joint, the joint aligner can also be used within other suitable joints within the human body.

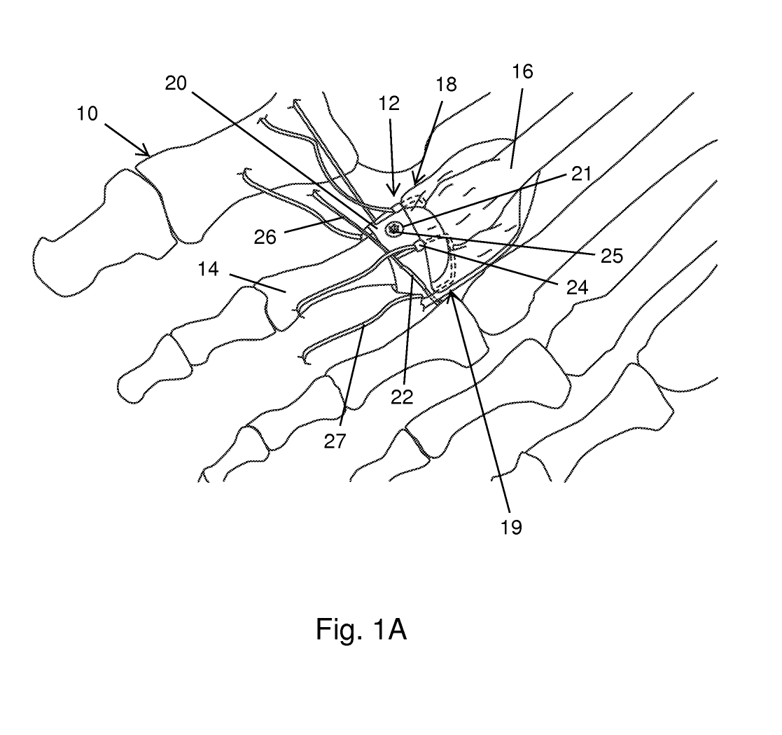

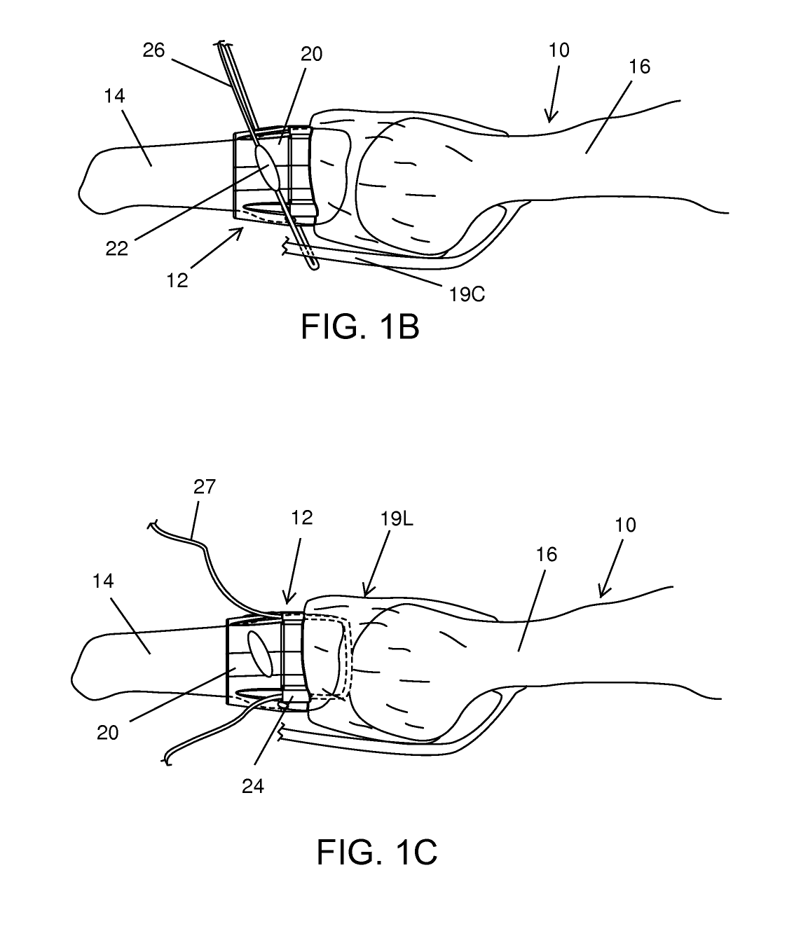

FIG. 1A is a perspective view of a portion of a foot 10 and an embodiment of a joint aligner implant 12 having features of the present invention implanted therein. In particular, FIG. 1A illustrates the joint aligner 12 being positioned about and/or secured to a first bone 14, i.e. a proximal phalanx in this particular example, substantially adjacent to a second bone 16, i.e. a metatarsal in this example, to maintain proper alignment between the metatarsal 16 and the proximal phalanx 14 within a joint 18, i.e. an MTP joint. Additionally, FIG. 1A further illustrates certain bone connecting material 19 that is positioned within and/or adjacent to the joint 18 and that is secured at least to the second bone 16. The bone connecting material 19 can include cartilaginous material 19C (illustrated in FIG. 1B), e.g., the plantar plate, and one or more ligaments 19L (illustrated in FIG. 1C).

As an overview, in certain embodiments, the joint aligner 12 is uniquely designed to enable the joint aligner 12 to quickly and easily fit over and around the proximal phalanx 14, and be effectively secured in such position, so that it can effectively align the proximal phalanx 14 relative to the metatarsal 16 within the MTP joint 18. Additionally, the joint aligner 12 can include unique design features such that the joint aligner 12, when appropriately and effectively positioned, enables easy suturing of the bone connecting material 19, i.e. of the cartilaginous material 19C and/or the ligaments 19L. With this design, any imperfections and abnormalities of the cartilaginous material 19C, e.g., the plantar plate, can be quickly and easily repaired, and any discomfort and deformities within and/or related to the MTP joint 18 can be effectively inhibited.

The design of the joint aligner 12 can be varied. For example, the joint aligner 12 can include an aligner body 20 having one or more fixation apertures 21 (only one is illustrated in FIG. 1A), one or more first connector apertures 22 (only one is illustrated in FIG. 1A), and one or more second connector apertures 24 (three are illustrated in FIG. 1A). Further, as shown in FIG. 1A, the aligner body 20 can be sized and shaped so as to be positioned about and/or secured to the proximal phalanx 14, e.g., substantially adjacent to the flare of the base of the proximal phalanx 14.

It should be noted that the use of the terms “first connector apertures” and “second connector apertures” is merely for purposes of illustration and ease of description, and any of the connector apertures 22, 24 can be referred to as the “first connector apertures” and/or the “second connector apertures”.

In some embodiments, the fixation aperture 21 is adapted to receive a fixation attacher 25, e.g., a screw, for securing the joint aligner 12 to the proximal phalanx 14.

Additionally, in certain embodiments, each of the one or more first connector apertures 24 is adapted to receive a flexible first connector 26, e.g., a suture, so that the first connector 26 can extend between the aligner body 20 and the cartilaginous material 19C to couple the proximal phalanx 14 to the cartilaginous material 19C. Somewhat similarly, in certain embodiments, each of the one or more second connector apertures 25 is adapted to receive a flexible second connector 27, e.g., a suture, so that the second connector 27 can extend between the aligner body 20 and a ligament 19L to couple the proximal phalanx 14 to the ligament 19L.

It should be noted that the use of the terms “first connector” and “second connector” is merely for purposes of illustration and ease of description, and any of the connectors 26, 27 can be referred to as the “first connector” and/or the “second connector”.

As described herein, the design of the joint aligner 12, i.e. the size, shape or dimensions of the various features of the aligner body 20, can be varied to suit the particular patient with whom the joint aligner 12 is used and/or to suit the particular procedures being performed.

Further, the joint aligner 12 can be made from any suitable material. For example, in various embodiments, the joint aligner 12 can be made from titanium, stainless steel, polyether ether ketone (PEEK), a cobalt-chromium alloy, or another suitable material.

FIG. 1B is a simplified side view of a portion of the foot 10, and the joint aligner 12 of FIG. 1A. In particular, FIG. 1B illustrates the aligner body 20 of the joint aligner 12 being positioned about and/or secured to the proximal phalanx 14 substantially adjacent to the metatarsal 16. Additionally, FIG. 1B further illustrates a first connector 26 that extends through one of the first connector apertures 22 of the aligner body 20 and to the cartilaginous material 19C to couple the proximal phalanx 14 to the cartilaginous material 19C.

In one embodiment, the aligner body 20 can include another first connector aperture 22 on the opposite side of the aligner body 20 (thus not being visible in FIG. 1B) and the first connector 26 can further extend from the cartilaginous material 19C to the other first connector aperture 22. In such embodiment, although not specifically illustrated in FIG. 1B, the first connector 26 can be tied dorsally after extending through one of the first connector apertures 22, to and through the cartilaginous material 19C, and then through the other first connector aperture 22. Further, in one exemplary application, the cartilaginous material 19C can comprise and/or include the plantar plate, and the first connector 26 can be utilized for repair of the plantar plate.

FIG. 1C is another simplified side view of a portion of the foot 10, and the joint aligner 12 of FIG. 1A. In particular, FIG. 1C illustrates the aligner body 20 of the joint aligner 12 being positioned about and/or secured to the proximal phalanx 14 substantially adjacent to the metatarsal 16. Additionally, FIG. 1C further illustrates a second connector 27 that extends through one of the second connector apertures 24 to one or more ligaments 19L to couple the proximal phalanx 14 to the ligaments 19L.

In one embodiment, as illustrated, the second connector 27 can be a horizontal mattress-type suture that subsequently extends back through another of the second connector apertures 24. In such embodiment, although not specifically illustrated in FIG. 1C, the second connector 27 can then be tied and thus secured in position. Alternatively, two second connectors 27 can be utilized with each second connector 27 extending just through one of the second connector apertures 24 and to the ligaments 19L. It should be noted that additional ligaments 19L can be coupled to the proximal phalanx 14 on the other side of the aligner body 20 utilizing one or more additional second connectors 27.

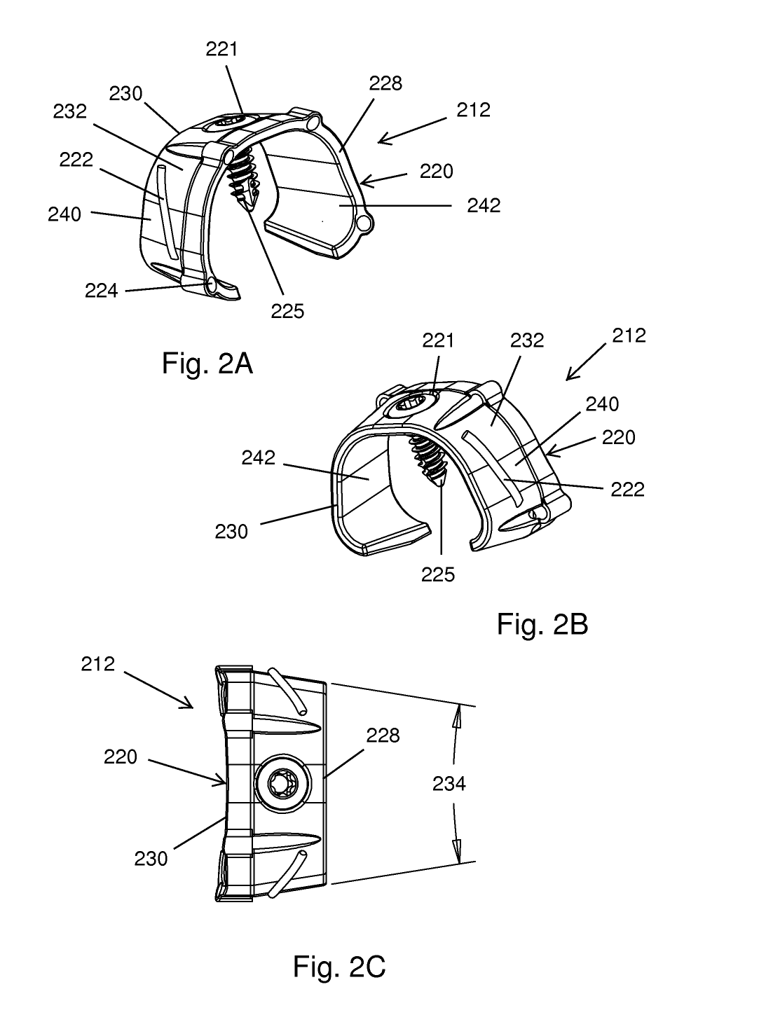

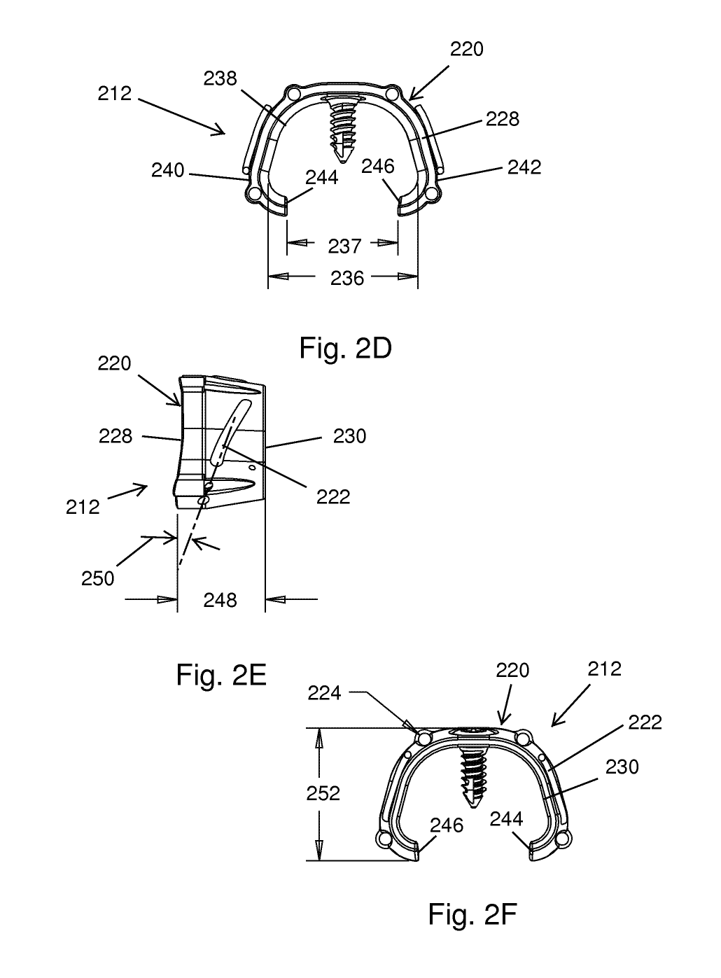

FIG. 2A is a perspective view of another embodiment of a joint aligner implant 212 having features of the present invention. As above, the design of the joint aligner 212, i.e. the size, shape or dimensions of the various features or aspects of the aligner body 220, can be varied to suit the particular patient with whom the joint aligner 212 is used and/or to suit the particular procedures being performed.

As shown in FIG. 2A, the joint aligner 212 again includes the aligner body 220 that is adapted to be positioned about and/or secured to the proximal phalanx 14 (illustrated in FIG. 1). Additionally, as shown, the joint aligner 212 includes a first end 228 and an opposed second end 230, with FIG. 2A providing a perspective view while looking generally at the first end 228 of the aligner body 220. It should be noted that the use of the terms “first end” and “second end” is merely for purposes of illustration and ease of description, and either end 228, 230 can be referred to as the “first end” and/or the “second end”.

Moreover, in this embodiment, the aligner body 220 again includes (i) a fixation aperture 221 that is adapted to receive a fixation attacher 225 for securing the joint aligner 212 to the proximal phalanx 14; (ii) one or more first connector apertures 222 that are each adapted to receive a first connector 26 (illustrated in FIG. 1A), e.g., a suture, to couple the joint aligner 212 and the proximal phalanx 14 to the cartilaginous material 19C (illustrated in FIG. 1B), e.g., the plantar plate, and/or for repair of the plantar plate; and (iii) one or more second connector apertures 224 that are each adapted to receive a second connector 27 (illustrated in FIG. 1A), e.g., a suture, to couple the joint aligner 212 and the proximal phalanx 14 to the ligament 19L (illustrated in FIG. 1C).

FIG. 2B is another perspective view of the joint aligner implant 212 of FIG. 2A. In particular, FIG. 2B illustrates a perspective view of the joint aligner 212 while looking generally at the second end 230 of the aligner body 220.

As shown in FIGS. 2A and 2B, in this embodiment, the aligner body includes two first connector apertures 222 that are positioned along and/or are projecting outward from an outer surface 232 of the aligner body 220. Additionally, the first connector apertures 222 are positioned on opposite sides of the aligner body 220, i.e. one first connector aperture 222 is positioned on a first side 240, e.g., a medial side, and one first connector aperture 222 is positioned on a second side 242, e.g., a lateral side, with only one of the first connector apertures 222 being visible in each of FIG. 2A and FIG. 2B. In particular, the first connector apertures 222 are tunnel-type apertures that are positioned substantially along the outer surface 232 of the aligner body 220 to enable any necessary repair to the cartilaginous material 19C, e.g., to the plantar plate. It should be noted that such first connector apertures 222 are usable as opposed to drilling holes through the bone material in the base of the proximal phalanx 14. Alternatively, the aligner body 220 can include a different number of first connector apertures 222 and/or the first connector apertures 222 can be positioned in a different manner on or about the aligner body 220. For example, in certain alternative embodiments, the aligner body 220 can include greater than two or less than two first connector apertures 222.

Additionally, in the embodiment illustrated in FIG. 2A, the aligner body 220 includes four second connector apertures 224 that are positioned along and/or are projecting outward from the outer surface 232 of the aligner body 220. Additionally, in this embodiment, two such second connector apertures 224 are positioned on the first (or medial) side 240 and the other two such second connector apertures 224 are positioned on the second (or lateral) side 242. Further, as shown, the second connector apertures 224 are tunnel-type apertures that are positioned substantially along the outer surface 232 of the aligner body 220. In one embodiment, the second connector apertures 224 are positioned near and/or adjacent to the first end 228 of the aligner body, and the second connector apertures 224 can be oriented substantially perpendicular to the first end 228 of the aligner body 220. Any combination of the second connector apertures 224 can be used for repair of transverse plane deformities. Moreover, it should be noted that these second connector apertures 224 can be placed far enough in the medial and lateral directions so as not to be prominent dorsally. Alternatively, the aligner body 220 can include a different number of second connector apertures 224 and/or the second connector apertures 224 can be positioned in a different manner on or about the aligner body 220. For example, in certain alternative embodiments, the aligner body 220 can include greater than four or less than four second connector apertures 224.

Further, as illustrated in FIGS. 2A and 2B, the aligner body 220 can be substantially C-shaped. With this design, the aligner body 220 can easily slide over the narrow portion of the shaft of the proximal phalanx 14 and then slide in the proximal direction, i.e. toward the metatarsal 16 (illustrated in FIG. 1), against the flare of the base of the proximal phalanx 14. Alternatively, the aligner body 220 can have a different shape.

Additionally, as shown in FIGS. 2A and 2B, the fixation aperture 221 can be substantially centrally positioned within the C-shaped aligner body 220. With this design, the fixation aperture 221 can be easily accessed for insertion of the fixation attacher 225 through the fixation aperture 221 and into the proximal phalanx 14. Alternatively, the fixation aperture 221 can be positioned differently than as illustrated in the Figures, and/or the aligner body 220 can include more than one fixation aperture 221.

FIG. 2C is a top view of the joint aligner implant 212 of FIG. 2A. As illustrated in FIG. 2C, the aligner body 220 includes the first (or proximal) end 228 (which is positioned closer to the metatarsal 16 (illustrated in FIG. 1) when the joint aligner 212 is positioned about the proximal phalanx 14 (illustrated in FIG. 1)), and the opposed second (or distal) end 230 (which is positioned farther from the metatarsal 16 when the joint aligner 212 is positioned about the proximal phalanx 14). Additionally, as shown, the aligner body 220 narrows or tapers from the first end 228 toward the second end 230 of the aligner body 220. This enables the joint aligner 212 to fit more comfortably and securely about the proximal phalanx 14 when the joint aligner 212 has been slid into proper position for attachment. In particular, in such embodiments, the aligner body 220 can have a taper angle 234 that illustrates the tapering of the aligner body 220 from the first end 228 toward the second end 230. The taper angle 234 can be varied to suit the specific shape of the bone, e.g., the proximal phalanx 14, to which the joint aligner 212 is attached. In certain embodiments, the aligner body 220 can have a taper angle 234 that is between approximately 12.0 and 24.0 degrees. For example, in certain non-exclusive alternative embodiments, the aligner body 220 can have a taper angle 234 of approximately 12.0, 14.0, 16.0, 18.0, 20.0, 22.0 or 24.0 degrees. Alternatively, the aligner body 220 can have a taper angle 234 that is greater than 24.0 degrees, less than 12.0 degrees, or some other value between 12.0 and 24.0 degrees.

FIG. 2D is an end view of the joint aligner implant 212 of FIG. 2A. In particular, FIG. 2D illustrates the view when looking at the first (or proximal) end 228 of the aligner body 220. Additionally, FIG. 2D further and more clearly illustrates that the aligner body 220 can be substantially C-shaped. Further, FIG. 2D also illustrates certain additional dimensions of the joint aligner 212, i.e. of the aligner body 220. For example, as shown in FIG. 2D, the aligner body 220 includes an outer spread 236 and an inner spread 237.

As illustrated, the outer spread 236 is the maximum spread (or distance) from an inner surface 238 of the first side 240 of the aligner body 220 to the inner surface 238 of the second side 242 of the aligner body 220. The outer spread 236 can be sized such that at least a portion of the inner surface 238 of the aligner body 220 can be positioned substantially directly adjacent to the proximal phalanx 14 (illustrated in FIG. 1) when the aligner body 220 is positioned against the flare of the base of the proximal phalanx 14. In certain embodiments, the outer spread 236 can be between approximately 0.40 inches and 0.55 inches. For example, in certain non-exclusive alternative embodiments, the outer spread 236 can be approximately 0.40, 0.42, 0.44, 0.46, 0.47, 0.49, 0.51, 0.53 or 0.55 inches. Alternatively, the aligner body 220 can be designed so that the outer spread 236 is greater than 0.55 inches, less than 0.40 inches, or some other value between 0.40 and 0.55 inches.

Additionally, as illustrated, the inner spread 237 is the spread (or distance) between a first tip 244 and a second tip 246 of the substantially C-shaped aligner body 220. The inner spread 237 can be sized such that the aligner body 220 is able to slide over the narrow portion of the shaft of the proximal phalanx 14, but also such that the tips 244, 246 can wrap somewhat around the proximal phalanx 14 to help secure the joint aligner 212 about the proximal phalanx 14 when the aligner body 220 is positioned against the flare of the base of the proximal phalanx 14. In certain embodiments, the inner spread 237 can be between approximately 0.30 inches and 0.40 inches. For example, in certain non-exclusive alternative embodiments, the inner spread 237 can be approximately 0.30, 0.31, 0.32, 0.33, 0.34, 0.35, 0.36, 0.37, 0.38, 0.39 or 0.40 inches. Alternatively, the aligner body 220 can be designed so that the inner spread 237 is greater than 0.40 inches, less than 0.30 inches, or some other value between 0.30 and 0.40 inches.

It should be noted that the use of the terms “first side” and “second side” is merely for purposes of illustration and ease of description, and either side 240, 242 can be referred to as the “first side” and/or the “second side”. Somewhat similarly, it should be noted that the use of the terms “first tip” and “second tip” is merely for purposes of illustration and ease of description, and either tip 244, 246 can be referred to as the “first tip” and/or the “second tip”.

FIG. 2E is a side view of the joint aligner implant 212 of FIG. 2A. In particular, FIG. 2E illustrates certain additional dimensions of the joint aligner 212, i.e. of the aligner body 220. For example, as shown in FIG. 2E, the aligner body 220 has a width 248 that extends from the first (or proximal) end 228 to the second (or distal) end 230. In certain embodiments, the aligner body 220 can have a width 248 of between approximately 0.20 inches and 0.35 inches. For example, in certain non-exclusive alternative embodiments, the width 248 of the aligner body 220 can be approximately 0.20, 0.22, 0.24, 0.26, 0.27, 0.28, 0.29, 0.31, 0.33 or 0.35 inches. Alternatively, the width 248 of the aligner body 220 can be greater than 0.35 inches, less than 0.20 inches, or some other value between 0.20 and 0.35 inches.

Additionally, FIG. 2E further illustrates that the first connector apertures 222 can be directed at an aperture angle 250 relative to the first end 228 of the aligner body 220. More specifically, the aperture angle 250 illustrated in FIG. 2E demonstrates that the first connector apertures 222 can be angled from dorsal distal to plantar proximal (e.g., when implanted about the proximal phalanx 14 (illustrated in FIG. 1)) to better enable proper and necessary repair to the plantar plate. In certain applications, the surgeon can repair and/or correct certain aspects of the cartilaginous material 19C (illustrated in FIG. 1B), e.g., the plantar plate, with first connectors 26 (illustrated in FIG. 1A) that extend through the first connector apertures 222. In another application, if there is a medial subluxation of a second digit, the surgeon would dissect the joint capsule from the base of the proximal phalanx 14, not the metatarsal head, and then repair the capsule with second connectors 27, i.e. either interrupted or mattress sutures, through the second connector apertures 224, i.e. through dorsal lateral and plantar lateral holes. This would give great stability to the MTP joint 18 (illustrated in FIG. 1) for the transverse plane deformities. Moreover, such repair and/or correction via the first connector apertures 222 can be used in combination with any other necessary or desired repair of the cartilaginous material 19C, e.g., the plantar plate.

In certain embodiments, such first connector apertures 222 can be directed at an aperture angle 250 of between approximately 15.0 and 25.0 degrees to ensure the proper positioning of certain of the first connectors 26. For example, in certain non-exclusive alternative embodiments, the aperture angle 250 of such first connector apertures 222 can be approximately 15.0, 16.0, 17.0, 18.0, 19.0, 20.0, 21.0, 22.0, 23.0, 24.0 or 25.0 degrees. Alternatively, such first connector apertures 222 can be directed at an aperture angle 250 that is greater than 25.0 degrees, less than 15.0 degrees, or some other value between 15.0 and 25.0 degrees.

FIG. 2F is another end view of the joint aligner implant 212 of FIG. 2A. In particular, FIG. 2F illustrates the view when looking at the second (or distal) end 230 of the aligner body 220. Additionally, FIG. 2F illustrates still yet other dimensions of the joint aligner 212. For example, FIG. 2F illustrates that the aligner body 220 has a height 252 that extends from the tips 244, 246 of the substantially C-shaped aligner body 220, i.e. the tips 244, 246 that define the inner spread 237 (illustrated in FIG. 2D), to the middle of the aligner body 220. In certain embodiments, the height 252 of the aligner body 220 can be between approximately 0.35 inches and 0.50 inches. For example, in certain non-exclusive alternative embodiments, the height 252 of the aligner body 220 can be approximately 0.35, 0.37, 0.39, 0.41, 0.42, 0.44, 0.46, 0.48 or 0.50 inches. Alternatively, the aligner body 220 can have a height 252 of greater than 0.50 inches, less than 0.35 inches, or some other value between 0.35 and 0.50 inches.

Additionally, FIG. 2F also illustrates that one or more of the first connector apertures 222 and/or the second connector apertures 224 can have a diameter of between approximately 0.03 inches and 0.05 inches. Alternatively, the first connector apertures 222 and/or the second connector apertures 224 can have a diameter that is greater than 0.05 inches or less than 0.03 inches. It should be noted that the diameter of the first connector apertures 222 and/or the second connector apertures 224 should be such as to enable the proper insertion of the first connectors 26 and/or the second connectors 27 (illustrated in FIG. 1A), e.g., sutures, through the first connector apertures 222 and/or the second connector apertures 224, respectively.

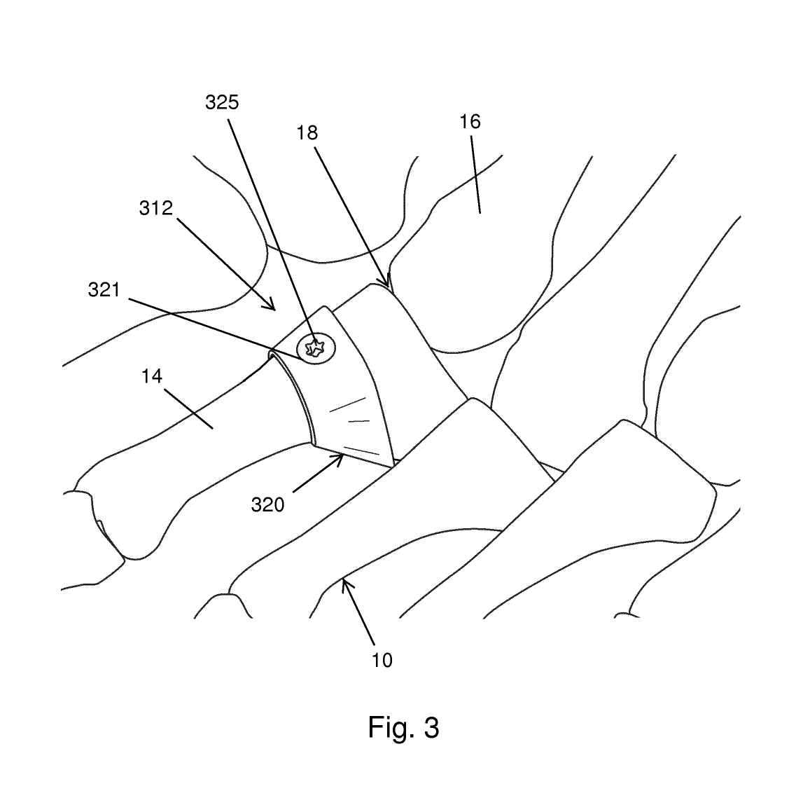

FIG. 3 is a perspective view of a portion of the foot 10 and yet another embodiment of a joint aligner implant 312 having features of the present invention implanted therein. As with the previous embodiments, the joint aligner 312 is illustrated as being positioned about and/or secured to the proximal phalanx 14 substantially adjacent to the metatarsal 16 to maintain proper alignment between the metatarsal 16 and the proximal phalanx 14 within the MTP joint 18.

The joint aligner 312 in this embodiment is somewhat similar to the joint aligners 12, 212 illustrated and described above. For example, as shown in FIG. 3, the joint aligner 312 again includes an aligner body 320 that is adapted to be positioned about the proximal phalanx 14, with the aligner body 320 including a fixation aperture 321 that is adapted to receive a fixation attacher 325 for attaching the joint aligner 312 to the proximal phalanx 14. However, in the embodiment illustrated in FIG. 3, the aligner body 320 is shown without any first connector apertures and/or second connector apertures.

While a number of exemplary aspects and embodiments of a joint aligner 12 have been shown and disclosed herein above, those of skill in the art will recognize certain modifications, permutations, additions and sub-combinations thereof. It is therefore intended that the joint aligner 12 shall be interpreted to include all such modifications, permutations, additions and sub-combinations as are within their true spirit and scope, and no limitations are intended to the details of construction or design herein shown.

What is claimed is:

1. A joint aligner for use with a flexible first connector for aligning a joint between a first bone and a second bone, the joint including bone connecting material that is connected to at least the second bone, the joint aligner comprising:
an aligner body that is adapted to be secured to the first bone, the aligner body including a first connector aperture that is adapted to receive the first connector so that the first connector can extend between the first connector aperture and the bone connecting material to couple the first bone to at least a portion of the bone connecting material.

2. The joint aligner of claim 1 wherein the aligner body includes a first end and an opposed second end, and wherein the first connector aperture extends at an aperture angle of between approximately fifteen and twenty-five degrees relative to the first end, the first connector aperture being adapted to receive the first connector so that the first connector can extend between the first connector aperture and the bone connecting material to couple the first bone to at least a portion of the bone connecting material.

3. The joint aligner of claim 1 wherein the aligner body includes a first end and an opposed second end, and wherein the first connector aperture extends substantially perpendicular to the first end, the first connector aperture being adapted to receive the first connector so that the first connector can extend between the first connector aperture and the bone connecting material to couple the first bone to at least a portion of the bone connecting material.

4. The joint aligner of claim 1 wherein the first connector aperture is positioned along an outer surface of the aligner body.

5. The joint aligner of claim 1 wherein the aligner body further includes a second connector aperture that is adapted to receive a flexible second connector so that the second connector can extend between the second connector aperture and the bone connecting material to couple the first bone to at least a portion of the bone connecting material, the second connector aperture being positioned along the outer surface of the aligner body.

6. The joint aligner of claim 1 wherein the aligner body further includes a second connector aperture that is adapted to receive the first connector so that the first connector can extend from the first connector aperture to the bone connecting material and to the second connector aperture to couple the first bone to at least a portion of the bone connecting material.

7. The joint aligner of claim 1 wherein the aligner body has a substantially C-shaped cross-section.

8. The joint aligner of claim 1 wherein the aligner body further includes a fixation aperture that is adapted for receiving a fixation attacher so that the fixation attacher can fixedly secure the aligner body to the first bone.

9. The joint aligner of claim 1 wherein the aligner body is tapered at a taper angle from a first end toward a second end.

10. The joint aligner of claim 9 wherein the taper angle is between approximately twelve and twenty-four degrees.

11. The joint aligner of claim 1 wherein the first bone is a proximal phalanx, the second bone is a metatarsal, and the joint is a metatarsophalangeal joint between the proximal phalanx and the metatarsal; wherein the bone connecting material is a plantar plate; and wherein when the aligner body is secured to the proximal phalanx, the first connector aperture is angled from dorsal distal to plantar proximal so that the first connector can extend between the first connector aperture and the plantar plate to couple the proximal phalanx to the plantar plate.

12. A combination including the joint aligner of claim 1, and a flexible first connector that extends between the first connector aperture and the bone connecting material to couple the first bone to at least a portion of the bone connecting material.

13. The combination of claim 12 wherein the aligner body further includes a second connector aperture that is adapted to receive the first connector, the first connector extending from the first connector aperture to the bone connecting material and back to the second connector aperture to couple the first bone to at least a portion of the bone connecting material.

14. A method for aligning a joint between a first bone and a second bone, the joint including bone connecting material that is connected to at least the second bone, the method comprising the steps of:
securing an aligner body to the first bone, the aligner body including a first connector aperture that is adapted to receive the first connector so that the first connector can extend between the first connector aperture and the bone connecting material to couple the first bone to at least a portion of the bone connecting material.

15. The method of claim 14 wherein the aligner body includes a first end and an opposed second end, and wherein the first connector aperture extends at an aperture angle of between approximately fifteen and twenty-five degrees relative to the first end, the first connector aperture being adapted to receive the first connector so that the first connector can extend between the first connector aperture and the bone connecting material to couple the first bone to at least a portion of the bone connecting material.

16. The joint aligner of claim 14 wherein the aligner body includes a first end and an opposed second end, and wherein the first connector aperture extends substantially perpendicular to the first end, the first connector aperture being adapted to receive the first connector so that the first connector can extend between the first connector aperture and the bone connecting material to couple the first bone to at least a portion of the bone connecting material.

17. The method of claim 14 further comprising the step of coupling the first bone to at least a portion of the bone connecting material with a flexible first connector that extends between the first connector aperture and the bone connecting material.

18. A joint aligner for use with a flexible first connector and a flexible second connector for aligning a joint between a first bone and a second bone, the joint including cartilaginous material and a ligament that are connected to at least the second bone, the joint aligner comprising:
an aligner body that is adapted to be secured to the first bone, the aligner body including: (i) a first end; (ii) an opposed second end; (iii) a first connector aperture that extends at an aperture angle of between approximately fifteen and twenty-five degrees relative to the first end, the first connector aperture that is adapted to receive the first connector so that the first connector can extend between the first connector aperture and the cartilaginous material to couple the first bone to the cartilaginous material; and (iv) a second connector aperture that extends substantially perpendicular to the first end, the second connector aperture being adapted to receive the second connector so that the second connector can extend between the second connector aperture and the bone connecting material to couple the first bone to at least the ligament.

19. The joint aligner of claim 18 wherein the aligner body further includes (i) a third connector aperture that is adapted to receive the first connector so that the first connector can extend from the first connector aperture to the cartilaginous material and to the third connector aperture to couple the first bone to the cartilaginous material; and (ii) a fourth connector aperture that is adapted to receive the second connector so that the second connector can extend from the second connector aperture to the ligament and to the fourth connector aperture to couple the first bone to the ligament.

20. A combination comprising the joint aligner of claim 18, a flexible first connector that extends between the first connector aperture and the cartilaginous material to couple the first bone to the cartilaginous material, and a flexible second connector that extends between the second connector aperture and the ligament to couple the first bone to the ligament.
